(19) United States
(12) Patent Application Publication
(10) Pub. No.: US 2005/0027278 A1
(43) Pub. Date: Feb. 3, 2005

(54) Absorbable article for mounting auxiliary pad

(75) Inventors: Satoshi Mizutani, Kagawa (JP); Koichi Yamaki, Kagawa (JP); Yuki Noda, Kagawa (JP)

Correspondence Address: DARBY & DARBY P.C., P. O. BOX 5257, NEW YORK, NY, 10150-5257, US

(73) Assignee: Uni-Charm Corporation, Kawanoe-Shi (JP)

(21) Appl. No.: 10/888,546

(22) Filed: Jul. 8, 2004

Related U.S. Application Data
(63) Continuation of application No. PCT/JP02/12016, filed on Nov. 18, 2002.

(51) Int. Cl. A61F 13/15; A61F 13/20

(52) U.S. Cl. 604/387

(57) ABSTRACT

The present invention concerns an absorbent article for attaching to the auxiliary pad capable of attaching an auxiliary pad detachably, and provides a hygienic product that can cope with the total menstruation and that can be handled easily.

A compressed groove (5) as guide element is formed on the body side face of a sanitary napkin (1), and an auxiliary pad (2) is attached by a pressure-sensitive adhesive (24) taking this compressed groove (5) as reference.

### CROSS-REFERENCE TO RELATED APPLICATIONS

This application is a Continuation of International Application No. PCT/JP2002/012016 filed Nov. 18, 2002, which application published in Japanese on Jun. 3, 2004 as WO 2004/045477 A1 under PCT Article 21 (2).

### FIELD OF THE INVENTION

The present invention relates to a hygienic article for attaching to the auxiliary pad that can attach to an auxiliary pad detachably.

### BACKGROUND ART

Conventionally, sheet-shaped absorbent articles enclosing an absorbent body are used widely, as a female sanitary article for body fluid absorption. Among these sheet-shaped absorbent articles, especially, for highly demanded sanitary napkins, a great deal of effort went into the prevention of menstrual blood from leaking.

As for sanitary napkins offering a high menstrual blood leakage preventive effect, for instance, the Japanese Utility Model Laid-Open No. 1983-191020 or the Japanese Utility Model Laid-Open No. 1983-43430 disclose those wherein a protrusion is formed in a portion in contact with the vicinity of the labia of the wearer, in order to enhance the adhesion of a sanitary napkin to the labial portion having a concave shape.

On the other hand, JP-A-1998-504486 proposes sanitary napkin whose fitness to the labial portion of the wearer, by bonding a columnar or cylindrical absorbent layer in a state placed horizontally to the skin abutment face side, for permitting the absorbent layer to follow easily the motion of the wearer.

However, the menstrual blood discharge amount is not constant during the total menstruation, but it varies; therefore, in the aforementioned conventional examples, the wearer ends up to feel a foreign object uselessly during a period where the menstrual blood discharge amount is not important.

On the other hand, a so-called ultra-thin type sanitary napkin of 1 to 4 mm in thickness dimension can certainly provide a comfortable feeling of wearing, but the wearer ends up by feeling uneasy about the leak of menstrual blood, when the menstrual blood discharge amount is important, because it fits bad to the labia portion.

Thus, conventional sanitary napkins are only appropriate either one of the case where the menstrual blood discharge amount is important or the case where it is small, and they can not cover the total menstruation by coping flexibly with the variation of menstrual blood discharge amount.

In this point, the Japanese Patent Laid-Open No. 1998-127692 discloses a stick-like auxiliary absorbent body to be used with a sanitary napkin. This auxiliary absorbent body fills the gap between the gorge of haunches and the napkin in order to enhance the effect of menstrual blood leak prevention, and can be used complementally when the menstrual blood discharge amount is important. Consequently, the wearer shall be relieved from a troublesome selection of an appropriate sanitary napkin.

Nevertheless, the fitting is complicated, because it is put directly in the haunches where menstrual blood has deposited, and so on, and especially, in case of affixing this auxiliary absorbent body to the napkin by a pressure sensitive adhesive, it is feared that it can not be affixed to an appropriate position, because it is difficult to apprehend an appropriate affixing position in respect to the napkin, therefore the fitness may lower, and the leakage prevention effect may not be deployed sufficiently.

The present invention has been made in view of inconveniences as mentioned above and has an object of providing a female hygienic capable of coping with the total menstruation, and easy to handle.

### DISCLOSURE OF THE INVENTION

In order to solve the aforementioned inconveniencies, the present invention is characterized by that an auxiliary pad complementing functions of body fluid absorption and so of an absorbent article is made detachable to the absorbent article, and a guide element for guiding the attachment position when the auxiliary pad is attached is afforded to the absorbent article.

More concretely, the present invention provides those mentioned below.

(1) An absorbent article for attaching to an auxiliary pad for assisting function for absorbing body fluid or other function of the absorbent article, comprising: a guide element for guiding an attachment position of said auxiliary pad on a body side face directed to a body side of a wearer when a wearer wears the absorbent article.

According to such absorbent article for attaching to the auxiliary pad of the present invention, an auxiliary pad for completing functions of body fluid (especially menstrual blood) absorption and so on of an absorbent article is made detachable and, at the same time, a guide element for guiding the auxiliary pad attachment position is formed on the body side face. As a result, the wearer can select to attach or not to attach the auxiliary pad to the absorbent article for attaching to the auxiliary pad, and in case of selecting to attach the auxiliary pad, the auxiliary pad can be adhered and fixed an appropriate position, taking the guide element as reference.

Here, the menstruation continues about one week or so, but a constant amount of menstrual blood is not always discharged as mentioned above. To be more specific, the menstrual blood is abundant from the first day to the third day or so of the menstruation, and about 70% of menstrual blood are discharged, but thereafter, remaining about 30% of menstrual blood are to be discharged slowly.

As the menstrual blood discharge amount varies, a sanitary napkin most appropriate for the wearer is necessarily different, following this. However, it is extremely troublesome for the wearer to select conveniently and buy such a sanitary napkin, and provokes a psychogenic burden.

In this respect, according to the present invention, during a period where menstrual blood discharge is abundant, the auxiliary pad may be applied following the guide element formed on the body side face of the absorbent article for attaching to the auxiliary pad and during the other period, the absorbent article for attaching to the auxiliary pad may be used as it is without attaching the auxiliary pad. In addition, by making the aforementioned absorbent article for attaching to the auxiliary pad a thin type of 1 to 4 mm in thickness dimension, a good wearing feeling can be obtained during the period where menstrual blood discharge is little.

Thus, according to the present invention, it is possible to cope flexibly with the wearer's demand which is different according to the variation of menstrual blood discharge amount, and the wearer turns up to be released from a trouble of buying beforehand various hygienic articles in order to cope with the variation of discharge amount, and using them as necessary, and able to use an appropriate hygienic article easily.

(2) The absorbent article for attaching to the auxiliary pad according to (1), wherein the guide element is provided linear symmetry taking a central line in the longitudinal direction as a symmetrical axis on the absorbent article for attaching to the auxiliary pad.

According to the absorbent article for attaching to the auxiliary pad of the present invention, guide elements for guiding the auxiliary pad attachment position are provided as a pair of lines right and left symmetrical in respect to the axe of the central line in the longitudinal direction. Therefore, the wearer can attach the auxiliary pad to an appropriate position on the absorbent article for attaching to the auxiliary pad, by attaching the auxiliary pad so that both side edges in the longitudinal direction of the auxiliary pad follow the guide element.

Thus, according to the present invention, as the wearer can attach the auxiliary pad correctly and rapidly to the absorbent article for attaching to the auxiliary pad, a hygienic article highly effective for preventing menstrual blood from leaking can be realized easily.

(3) The absorbent article for attaching to the auxiliary pad according to (1) or (2), wherein said guide element is a continuous line or dashed line of an oval shape having a central line in the longitudinal direction of said absorbent article for attaching to the auxiliary pad as a major axis thereof.

In the absorbent article for attaching to the auxiliary pad according to the present invention, the guide element is made as continuous line or dashed line, and not as a pair of right and left symmetrical lines as in the aforementioned (2). Consequently, the attachment of auxiliary pad can further be facilitated, because the wearer may attach the auxiliary pad so as to contain in the area inside this continuous line or dashed line.

Especially, in case where a plurality of guide elements are formed as in (5) mentioned below, if the guide element is a pair of lines, there is a danger of mistaking the attachment position if the pairing is not confirmed; however, in the present invention where the area to which the auxiliary pad is shown by surrounding with the guide element made of continuous line or dashed line, the attachment position can be apprehended easily, so the danger of mistaking the auxiliary pad attachment position is extremely low.

The “continuous line” and “dashed line” are preferably “substantially same dimensions as dimensions of the auxiliary pad” to “dimensions of the order of exceeding the attached auxiliary pad by about 3.0 mm”. Within this range, especially preferable dimensions in consideration of the positional relation with the labia are dimensions of the order of exceeding the attached auxiliary pad by about 1.0 to 3.0 mm.

(4) The absorbent article for attaching to the auxiliary pad according to any one from (1) to (3), wherein said guide element comprises a compressed line, a compressed groove, a colored sheet or a printed sheet formed on the body side face of said absorbent article for attaching to the auxiliary pad.

In the absorbent article for attaching to the auxiliary pad according to the present invention, the guide element for guiding the auxiliary pad attachment position comprises any one of compressed line, compressed groove, colored sheet or printed sheet.

In case of comprising the guide element with compressed line or compressed groove, the wearer can recognize by view the pattern formed by compression and different from non-compressed portion as the auxiliary pad attachment position and in addition to this, it comes to have an effect of limiting the diffusion of the menstrual blood by the presence of compressed line or compressed groove, even if the menstrual blood absorbed by the auxiliary pad flows out from the auxiliary pad to the absorbent article for attaching to the auxiliary pad.

Such compressed line or compressed groove is formed by molding integrally a liquid permeable surface side sheet position on the body side face of the absorbent article for attaching to the auxiliary pad and an absorbent body position of the opposite side to the body side thereof.

It should be noted that the width dimension of a compressed line or a compressed groove as a guide element is necessary to be set in a range that can be recognized by eyes, and it is preferable to be set to the dimension of (6) or (7) mentioned below.

In case where the guide element is composed of colored sheet or printed sheet, the area appropriate for attaching the auxiliary pad and the inappropriate are will be different in color. Consequently, the wearer comes to be able to distinguish them easily, allowing to plan to make the auxiliary pad attachment operation more rapid.

Hydrophilic and well-drained materials can be used for colored sheet or printed sheet. For instance, thermal bond non-woven fabric made of synthetic fiber subjected to the hydrophilic treatment can be cited as example. Cellulose fiber may be blended in this non-woven fabric in a range 30% or less.

As for coloring method, method for coloring the fiber itself by blending colorant in the raw material resin of the fiber, method for printing pigments on a sheet, method of transferring hot melt adhesive blended with pigments to a sheet, and so on can be cited.

The color tone is preferably made blue, green, yellow, orange, dark blue, and so on. This is the consideration of cleanness.

(5) The absorbent article for attaching to the auxiliary pad according to any one from of (1) to (3), wherein said guide element comprises one of the same kind selected from a group consisting of compressed line, compressed groove, colored sheet and printed sheet formed on the body side face of said absorbent article for attaching to the auxiliary pad, or combination of one or more different kind selected from said group.

In such absorbent article for attaching to the auxiliary pad according to the present invention, a plurality of guide elements is disposed in different positions respectively. Therefore, even when the wearer uses absorbent articles of different size, the absorbent article can be attached following a guide element corresponding to the respective size.

The following cases are supposed as cases where the wearer uses the aforementioned hygienic article selectively as a sanitary product. Namely, \mathring{1} variation of the menstrual blood discharge amount according to the menstrual cycle, \mathring{2} variation of the menstrual blood leakage due to the motion during the wearing (for instance, menstrual blood tends to flow to the back and leak during the sleep, and so on), \mathring{3} variation of the menstrual blood discharge amount according to a certain experience (for instance, the menstrual blood discharge amount is more important for primiparae and multiparae compared to nulliparae, and so on), (4) change of figure along with aging (for instance, 30 years old or more, especially haunches tend to drop along with the weakening metabolism, and the contact position with the product turns up to be different, and so on) or other can be cited.

As mentioned above, menstrual blood discharge amount or leakage are not uniform; therefore, the sense of security that the wearer feels by her bodily sensation turns up to be different according to the menstrual blood discharge amount, figure, and so on. Consequently, it is preferable that there is a plurality of sizes for the auxiliary pad, so as to differentiate the body fluid absorption capability. In addition, it is preferable that there is a plurality of attachment positions for the auxiliary pad in order to cope with the size impression according to different flow directions of menstrual blood and figure.

In this point, according to the present invention, as a plurality of guide elements is disposed, it becomes possible to execute a fine adjustment for the performance of the absorbent article for attaching to the auxiliary pad, by using auxiliary pads different in size, or changing the attachment position thereof, even if a common absorbent article for attaching to the auxiliary pad is used.

As for the size of auxiliary pad, it is preferable that there are at least two kinds of small size/large size and more preferably, three kinds of small size/medium size/large size.

The overall length of a concrete auxiliary pad is preferable set in a range of 60 to 80 mm for small size, 80 to 120 mm for medium size, and 110 to 150 mm for large size.

Also, for the guide element to be disposed on the absorbent article for attaching to the auxiliary pad, at least two, or three elements are to be formed so as to cope with such a variety of auxiliary pads.

There, it is preferable to set the dimensions of this guide element in a range of “dimensions substantially of the same order as the overall length of the auxiliary pad” to “dimensions of the order of exceeding the auxiliary pad by about 3.0 mm”. To be more concrete, it is preferable that the guide element is in a range of “60 to 63” to “80 to 83” mm in case where the overall length of the auxiliary pad has a length dimension of 60 to 80 mm, “80 to 83” to “120 to 123” mm in case where the overall length of the auxiliary pad has a length dimension of 80 to 120 mm, and “110 to 113” to “150 to 153” mm in case where the overall length of the auxiliary pad has a length dimension of 110 to 150 mm. As the result, a plurality of parabolas of the similar figure will be formed on the body side face of the absorbent article for attaching to the auxiliary pad.

It should be noted that the preferable width dimension of the guide element is set in a range of “dimension substantially of the same order as the width dimension of the auxiliary pad” to “dimension of the order of exceeding the width dimension of the auxiliary pad by about 3.0 mm”. To be more concrete, it is preferable to set in a range of “15 to 18” to “60 to 63” mm.

The whole size of the absorbent article for attaching to the auxiliary pad is required to be sufficiently large for attaching the aforementioned auxiliary pad. More concretely, it is in a range to have a overall length of 130 to 350 mm, preferable a range of 190 to 300 mm and especially preferably a range of 210 to 260 mm. Besides, the width dimension of the absorbent article for attaching to the auxiliary pad is preferably in a range of 60 to 120 mm (except for wings, if they are provided) and more preferably in a range of 65 to 100 mm.

As cases where the position of the guide element is changed according to the menstrual blood flow direction, for instance, a case where menstrual blood tends to leak excessively to the backward of the haunches during the sleep and so on can be cited. In such a case, the guide element is formed in the backward area (area in contact with the vicinity of the anus of the wearer during the wearing), to cope with backward menstrual blood leakage. More concretely, the length of the absorbent article for attaching to the auxiliary pad is set to 250 to 350 mm so as to attain the vicinity of the sacrum, and the guide element is provided in the backward area of such absorbent article for attaching to the auxiliary pad. It should be noted that, it is preferable to form in a range of 60 to 120 mm in width dimension so as not to deteriorate the wearing feeling.

The plurality of guide elements is all composed of the same kind (for instance, all guide elements are consist of compressed line, and so on), in addition, they can also be composed by combining different kinds thereof conveniently (for instance, guide elements are composed by combining a compressed line and a compressed groove, and so on).

(6) The absorbent article for attaching to the auxiliary pad according to (4) or (5), wherein said compressed line has a width dimension ranging from 1.0 to 5.0 mm.

In such absorbent article for attaching to the auxiliary pad according to the present invention, the wearer comes to be able to distinguish this compressed line easily, because the width dimension of the compressed line is set in a range of the order of 1.0 to 5.0 mm. As a result, the wearer can apprehend easily the auxiliary pad attachment position.

(7) The absorbent article for attaching to the auxiliary pad according to (4) or (5), wherein an interval dimension of a cavity in said compressed groove is 0.5 to 1.5 mm while an interval dimension of a convex portion in said compressed groove is 0.5 to 1.5 mm.

In such absorbent article for attaching to the auxiliary pad according to the present invention, the interval dimension of the concave portion of the compressed groove constituting the guide element is set to 0.5 to 1.5 mm, and the interval dimension of the convex portion 0.5 to 1.5 mm. Consequently, the wearer can apprehend the compressed groove visually and easily, allowing to facilitate the attachment operation of an auxiliary pad to the absorbent article for attaching to the auxiliary pad.

(8) The absorbent article for attaching to the auxiliary pad according to (4) or (5), wherein said colored sheet is enclosed visually recognizably in the absorbent article for attaching to the auxiliary pad.

According to the present invention, the colored sheet is not arranged on the body side face of the absorbent article for attaching to the auxiliary pad, but enclosed in the absorbent article for attaching to the auxiliary pad. To be more specific, it is arranged between a liquid permeable surface side sheet positioned on the body side of the absorbent article for attaching to the auxiliary pad and an absorbent body positioned on the opposite body side face to body side face of the surface side sheet.

As a result, the colored sheet will not be peel off partially from the surface side sheet and crinkled by the wearing pressure or rubbing generated during the wearing, and colored sheets will not overlay each other, as in case where the colored sheet is arranged on the body side face of the surface side sheet, and a situation where the colored sheet inhibits the liquid permeability of the surface side sheet is prevented.

It should be noted that, in the present invention, the wearer recognizes the color of the colored sheet through the liquid permeable surface side sheet.

(9) An auxiliary pad to be attached to the absorbent article for attaching to the auxiliary pad according to any one of (1) to (8), comprising: a guided element corresponding to the guide element included in said absorbent article for attaching to the auxiliary pad.

The auxiliary pad according to the present invention is provided with a guided element, in correspondence to the absorbent article for attaching to the auxiliary pad comprising the guide element. This guided element may be one capable of attaching an auxiliary pad to the absorbent article for attaching to the auxiliary pad and, typically, pressure-sensitive adhesives can be cited.

The area for application of this pressure-sensitive adhesive is not limited specially, and for example, in case of an auxiliary pad of band-like shape, it may be applied all over the surface, in line shape, network shape, dot shape, or the like on the face side in contact with the absorbent article for attaching to the auxiliary pad, or a sheet with pressure-sensitive adhesive where pressure-sensitive adhesive is applied in the aforementioned pattern may be pasted.

As for sheet for adhesive application, film, foil paper, non-woven fabric sheet, and so on can be used. The sheet to which adhesive is applied is formed with dimensions in a range equivalent to the setting area of the guide element to be formed on the body side face of the absorbent article for attaching to the auxiliary pad, or a range not exceeding the setting area of the guide element.

The adhesive strength of the pressure-sensitive adhesive is at least 10000 Pa in count value by the pikumatatsuku tester and it is preferable to provide with an adhesive strength in a range of 10000 to 50000 Pa. The danger of dropping during the use is prevented, by affording an adhesive strength in such range.

Concerning the pressure-sensitive adhesive, it is preferable to use a pressure sensitive hot melt adhesive, in consideration of the application stability also. As for the pressure-sensitive type hot melt adhesive presenting a good application stability, one obtained by melting and blending 15 to 25 mass % of SEBS (styrene-ethylene/butadiene-styrene block copolymer), 15 to 35 mass % of plasticizer and 40 to 70 mass % of tackifier can be cited. For this pressure sensitive type hot melt adhesive, anti-oxidant or anti-fluorescent agents can be added in a range of 0.1 to 1.0 mass %. Thus obtained adhesive is coated by means of a hot melt applicator in a range of 50 to 150 g/m² by a specific weight per unit of.

(10) The auxiliary pad according to (9), wherein said auxiliary pad has a band shape or flat shape.

In the auxiliary pad according to the present invention, its shape constitutes a band-like or flat shape.

As for the size of such auxiliary pad, at least a size adapting to the vicinity of the labia is necessary, and it is preferable to set the whole length dimension in the longitudinal direction in a range of 60 to 150 mm and, more preferably in a range of 80 to 130 mm.

In case of a band-like auxiliary pad, the dimension in the lateral direction is preferably in a range of 15 to 60 mm and more preferably in a range of 25 to 40 mm. Besides, the height dimension is preferably in a range of 5 to 30 mm and more preferably in a range of 10 to 20 mm.

It should be noted that, in the present invention, “band-like” means that the auxiliary pad is band-like in the longitudinal direction, view from the body side face and the shape of its section may be circular, oval, triangular, square or polygonal more than square or other shapes that adhere conveniently to the labia of the wearer.

(11) The auxiliary pad according to (10), wherein said flat shape auxiliary pad is set to have a size so as to generate a surplus in the flat shape auxiliary pad when the guide element included in the absorbent article for attaching to an auxiliary pad is matched with the guided element included in said flat shape auxiliary pad in correspondence to the guide element, and said flat shape auxiliary pad is designed to be attached to said absorbent article for attaching to the auxiliary pad by folding during the use.

In the auxiliary pad according to the present invention, the interval dimension of the guided element in the auxiliary pad is longer than the interval dimension of the guide element in the absorbent article for attaching to the auxiliary pad. Therefore, when the auxiliary pad is attached, the auxiliary pad shall be folded in a way to protrude to the body side.

Thus, since the auxiliary pad is folded before attaching, it comes to have the following three effects. Namely, \mathring{1} since the auxiliary pad has a shape aligned with the direction of the labia beforehand, it copes previously with the menstrual blood flowing out along the shape of the labia. \mathring{2} Since the protrusion is formed by simply folding, it can deform appropriately according to the labial shape of the wearer by the wearing pressure. \mathring{3} Even in case where the auxiliary pad attachment position is wrong (for instance, in case of slightly slant state, and so on), the wearer turns up to be directed to correct the position immediately, as the wearer can feel easily a bad wearing feeling.

For the flat shape auxiliary pad, the width dimension of the base when folded is preferably in a range of 25 to 40 mm. In this case, the maximum height dimension at the summit is set to vary in a range of 10 to 20 mm.

In case where the guided element of the auxiliary pad in the present invention is an pressure-sensitive adhesive, it is preferable to provide an application area of pressure-sensitive adhesive in the vicinity of the side edge portion of the inner wall face of the folded auxiliary pad and at the same time, to fold further the auxiliary pad so that the application area of pressure-sensitive adhesive is directed to the body side face of the absorbent article for attaching to the auxiliary pad (refer to FIG. 4). By doing this, an unintended adhesion of adhesive each other during the operation of folding and attaching the auxiliary pad can be prevented, and the application area comes easily into contact with the absorbent article for attaching to the auxiliary pad.

The area to be coated with adhesive is provide at least in the vicinity of the side edge portion of the auxiliary pad, it is applied in dimensions throughout the full end in the longitudinal direction of the folded auxiliary pad, in a range of 1 to 5 mm in width, equal or superior to 10 mm, preferably 30 to 120 mm, more preferably 40 to 90 mm in length dimension.

It is also possible to bond previously two-folded inner sides each other by an adhesive, and prevent the discrepancy of folding position according to the wearer.

(12) The auxiliary pad according to (11), wherein said flat shape auxiliary pad has a folding guidance element indicating a folding position.

In the auxiliary pad according to the present invention, a folding guidance element is disposed at a position for folding the auxiliary pad. Therefore, the wearer can fold the auxiliary pad easily along this folding guidance element.

The “folding guidance element” is designed to indicate the folding position of the auxiliary pad, in order to facilitate to engage the auxiliary pad in the folded state with the pudendal slit of the wearer, when the auxiliary pad is folded. Consequently, it may drawn at any position on the auxiliary pad, provided that it indicates a folding position that would facilitate to engage the folded auxiliary pad in the folded state with the pudendal slit.

As an advantageous example thereof, for instance, it may be a line drawn in the substantially central line portion in the longitudinal direction, or a line drawn in the substantially central line portion in the lateral direction. The aforementioned line may be offset, or, it may be provided in imbalance according to the labial shape of the wearer, provided that the aforementioned object, function and effect can be deployed, in this portion. In the preferred embodiment, it is provided in the middle area of the auxiliary pad, and more preferably, along the vertical central line.

The folding guidance element is not limited to a simply drawn line, it may also be one took to fold previously, and it may also be formed by weakening the strength of the summit by affording a break line with a line or a broken line, or arranging absorbent bodies divided with an interval in a range of 1 to 5 mm in width dimension around the summit. It should be noted that the folding guidance element may be continuous in a streak, or intermittent.

The presence of such folding guidance element facilitates, to fold along the folding guidance element, allowing to plan to make the attachment operation to the absorbent article for attaching to the auxiliary pad more rapid.

(13) The auxiliary pad according to any one of (9) to (12), wherein an adhesive is applied to the skin abutment face of said auxiliary pad.

In the auxiliary pad according to the present invention, an adhesive is applied to the skin abutment face. Therefore, it becomes possible to adhere the auxiliary pad closely to the haunches of the wearer, and a gap is prevented from generating between the auxiliary pad and the body, even if the wearer changes suddenly her posture. Thus the wearer can perform a free activity in peace, without being constricted in her behavior.

This adhesive can be adhered to the skin, and on the other hand, should not afford an excessive adhesive strength. To be more concrete, as for the adhesive strength of the adhesive, the value of 180 degree peel strength measured by a traction rate of 100 m/min, after pressure boding of pressure-sensitive adhesive put on a polyester film with a roll of 3000 Pa in load on a stainless steel board is preferably in a range of 100 to 2000 mN, and the shearing strength in a range of 3000 to 15000 mN. This is in consideration of the burden to the skin.

As for an adhesive that can be used in the present invention, gel adhesive or the like comprising water-soluble polymer, crosslinking agent, plasticizer, and moisture can be cited. To be more specific, examples of water-soluble polymer comprise gelatin, sodium polyacrylate, polyvinyl alcohol, carboxylmethyl cellulose, and so on, examples of crosslinking agent include calcium chloride, magnesium sulfate or other water-soluble metallic salts, and examples of plasticizer comprise glycerin, wax, paraffin, or others.

Other than these, pressure sensitive hot melt adhesives also can be used. The pressure sensitive hot melt adhesive can be obtained by using styrene-ethylene/butadiene-styrene block copolymer (SEBS), styrene-isoprene-styrene block copolymer (SIS), styrene-butadiene-styrene block copolymer (SBS), styrene-ethylene/propylene-styrene block copolymer (SEPS) or other synthetic rubber resins as main ingredient, and melting and blending therein terpene resin, rosin resin or other tackifiers and wax or other plasticizers.

Further, silicone resin type adhesives can also be used. As the silicone resin type adhesive, the mixture comprising mainly silicone resin and fluorocarbon resin, made by mixing crosslinking agent of platinum, molybdenum, antimony or other metal salt and plasticizer such as ester type wax, glycerin, machine oil or the like.

Thus, various kinds of adhesives that can be used for the present invention exist; however, considering the application stability, it is preferable to use a pressure sensitive hot melt adhesive. As for the pressure sensitive hot melt adhesive presenting a good application stability, one obtained by melting and blending 15 to 25 mass % of SEBS, 15 to 35 mass % of plasticizer and 40 to 70 mass % of tackifier can be cited. For this pressure sensitive hot melt adhesive, anti-oxidant or anti-fluorescent agents can be added in a range of 0.1 to 1.0 mass %.

It is also possible to make the adhesive elastic, and reduce the stress to the skin, by introducing nitrogen gas during the application of the aforementioned adhesive, and blending nitrogen gas with the adhesive, to make an adhesive elastic.

(14) The auxiliary pad according to any one of (9) to (13), wherein said auxiliary pad is an auxiliary pad for incontinence of urine.

By intermediate of the auxiliary pad according to the invention, it can be used as absorbent pad of countermeasure for incontinence. In short, as the ostium vaginae discharging menstrual blood and the urethral meatus for discharging urine are both found between interlabia, urine can be absorbed, in case of using the auxiliary pad according to the present invention pinched between interlabia.

Thus, according to the present invention, an absorbent pad effective for incontinence, especially for a slight incontinence can be obtained, as it can absorb urine between interlabia, especially near the urethral meatus.

(15) The auxiliary pad according to any one of (9) to (13), wherein said auxiliary pad is for absorbing the vaginal discharge.

By intermediate of such auxiliary pad according to the present invention, the auxiliary pad can be used for absorbing the vaginal discharge. In short, as the auxiliary pad according to the present invention is pinched between interlabia for use, it can absorb the discharge other than menstrual blood from the ostium vaginae (discharge), so it can be used for this application (vaginal discharge absorption).

(16) A hygienic product: comprising the absorbent article for attaching to the auxiliary pad according to any one of (1) to (8), and an auxiliary pad having a guided element corresponding to the guide element included in the absorbent article for attaching to an auxiliary pad, wherein said absorbent article for attaching to the auxiliary pad and said auxiliary pad can be used themselves independently and, at the same time, can be used by freely combining both.

According to the present invention, an absorbent article for attaching to the auxiliary pad and an auxiliary pad to be attached to the same constitute a set. Consequently, the wearer can assembly a most appropriate hygienic article, provided that she has a set of hygienic article according to the present invention.

As a result, the wearer turns up to be released from both of troubles of buying beforehand various hygienic articles, and using them by selecting the most appropriate one as necessary.

(17) The hygienic product of (16), wherein said auxiliary pad comprises a plurality of auxiliary pads having different body fluid absorption capacity, and said guide element is provided according to the number of said auxiliary pad.

According to the present invention, a plurality of auxiliary pads different in size and an absorbent article for attaching to the auxiliary pad having a plurality of guide elements corresponding to them constitute a set. Therefore, it becomes possible for the wearer to execute a fine adjustment according to the body fluid discharge amount of the day, or her figure, leakage tendency of menstrual blood or other body fluid.

Thus, the hygienic article set according to the present invention can be used as hygienic article having versatile performances, different from ready-made absorbent products.

### BRIEF DESCRIPTION OF THE DRAWINGS

FIG. 1 is a schematic top view showing a sanitary napkin as an absorbent article for attaching to the auxiliary pad according to the present embodiment;

FIG. 2 is a cross sectional view along the line A-A in FIG. 1;

FIG. 3 is a schematic top view showing an auxiliary pad according to the present embodiment;

FIG. 4 is a cross sectional view along the line B-B in FIG. 3;

FIG. 5 is a schematic perspective view showing a state where the auxiliary pad is attached to the sanitary napkin according to the present embodiment;

FIG. 6 is a cross sectional view along the line C-C in FIG. 5;

FIG. 7 is a schematic top view showing a shape of a guide element provided on the sanitary napkin;

FIG. 8 is an explanatory illustration explaining the sanitary napkin provided with a colored sheet colored by a color different from the surface side sheet;

FIG. 9 is a top view showing the sanitary napkin where a plurality of guide elements is formed;

FIG. 10 is top view showing the sanitary napkin with wing where a plurality of guide elements is formed;

FIG. 11 is a schematic perspective view showing the auxiliary pad with triangular cross section;

FIG. 12 is a longitudinal sectional view showing a state where an adhesive is pasted to the body side face of the auxiliary pad.

### DETAILED DESCRIPTION OF THE PREFERRED EMBODIMENT

Now, an embodiment of absorbent article for attaching to the auxiliary pad of the present invention shall be described referring to drawings.

[Basic Composition of the Present Embodiment]

<Absorbent Article for Attaching to the Auxiliary Pad (Sanitary Napkin)>

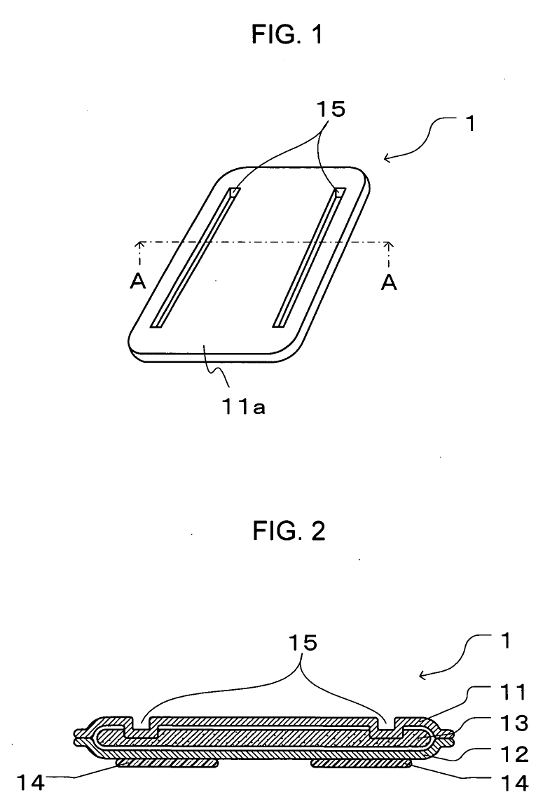

First, a sanitary napkin as absorbent article for attaching to the auxiliary pad in the present embodiment shall be described. FIG. 1 is a schematic top view showing a sanitary napkin 1 according to the present embodiment and FIG. 2 is a cross section along the line A-A in FIG. 1.

As shown in FIG. 1, the sanitary napkin 1 according to the present embodiment has a substantially longitudinal flat shape, in appearance shape thereof. Then, two long shape compressed grooves 15 are formed along the longitudinal direction. This compressed groove 15 has become guide element that will constitute the reference of attachment position for an auxiliary pad not illustrated here.

This sanitary napkin 1 has a structure where a flat shape absorbent body 13 for absorbing body liquid (especially menstrual blood) is sandwiched by a liquid permeable surface side sheet 11 disposed on the body side face for filtering liquid and a liquid-impermeable back face side sheet 12 disposed on the [symbol] garment side face to body side face and impermeable to liquid, and a adhesive 14 for fixing to an underwear is applied to the garment side of the liquid-impermeable back face side sheet 12, as shown in FIG. 2.

A compressed groove 15 is formed on the body side face of the sanitary napkin by molding integrally the liquid permeable surface side sheet 11 and the absorbent body 13. Since it is possible to position by matching the position of a side edge portion of (not shown) auxiliary pad with the compressed groove 15, the attachment operation of the auxiliary pad becomes rapid and sure for the wearer.

<Auxiliary Pad>

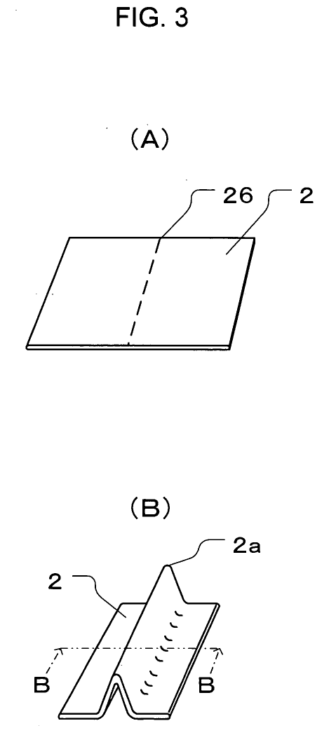

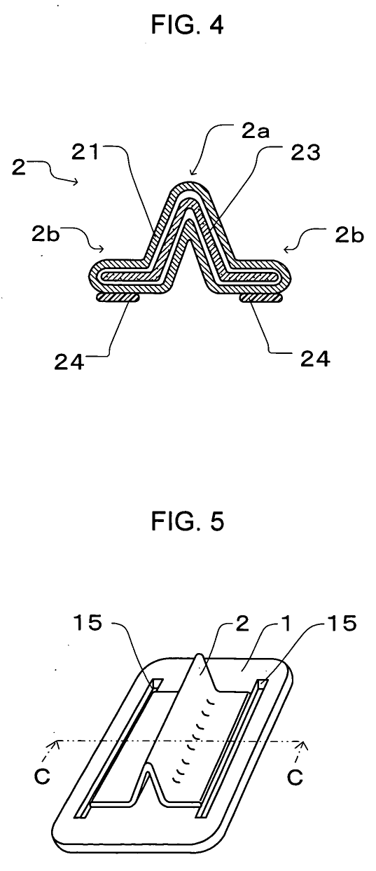

Next, the auxiliary pad used for the present embodiment shall be described. FIG. 3 is a schematic top view showing the auxiliary pad of the present embodiment and FIG. 4 is a cross section along the line B-B in FIG. 3(B).

The appearance shape of the auxiliary pad 2, before attachment to the sanitary napkin, is flat shape as show in FIG. 3(A). Then, a break line 26 which is a folding guidance element is disposed in the middle thereof. Consequently, the wearer can fold the auxiliary pad 2 easily following this break line 26, thereby, forming a convex area 2a as shown in FIG. 3(B).

The auxiliary pad 2 comprises, as shown in FIG. 4, an absorbent body 23 for absorbing menstrual blood or other body fluid, and a liquid permeable cover sheet 21 covering this absorbent body 23 in a way to envelop the whole, and the top face is made as skin abutment face in contact with the skin of the wearer and, at the same time, the bottom face side is made as attachment face to be attached to the sanitary napkin 1.

An adhesive 24 which is a guided element for affixing the auxiliary pad 2 to a (not shown) sanitary napkin is applied to two points along the longitudinal direction of the vicinity of both edge portions. This adhesive 24 will be position on the opposite body side face to body side face of the flat area 2b which is an area other than the convex area 2a, when the auxiliary pad 2 is folded so as to be convex to the body side following the aforementioned break line 26. Therefore, the adhesive comes easily into contact with the sanitary napkin and, at the same time, the danger that the present embodiment 24 is adhered each other is reduced.

<Attachment State of Sanitary Napkin and Auxiliary Pad>

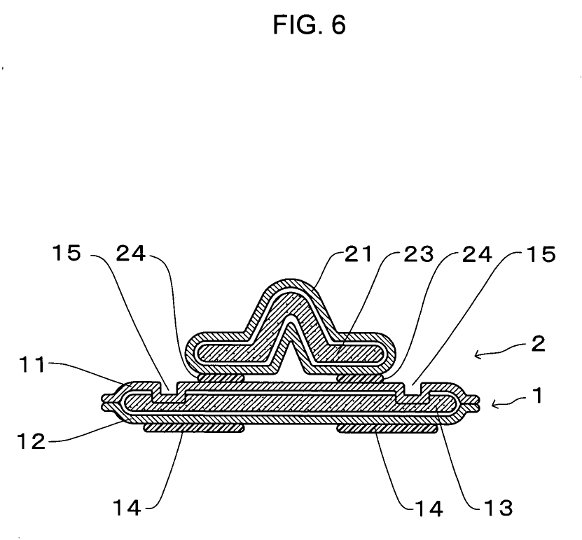

Next, the attachment state of a case where the auxiliary pad 2 is attached to the aforementioned sanitary napkin 1 for use shall be described. FIG. 5 is a schematic perspective view showing a state where the auxiliary pad 2 is attached to the sanitary napkin 1, and FIG. 6 is a cross section along the line C-C in FIG. 5.

The auxiliary pad 2 is, as shown in FIG. 5, attached to the body side face of the sanitary napkin 1 to be fixed to the underwear, and used by the wearer in this state.

The wearer can attach the auxiliary pad 2 to the sanitary napkin 1 easily through the pressure-sensitive adhesive 24, by matching right and left both side end of the auxiliary pad 2 to respective compressed groove 15, because two compressed grooves 15 are formed, as shown in FIG. 6, on the body side face of the sanitary napkin 1.

[Guide Element of Sanitary Napkin]

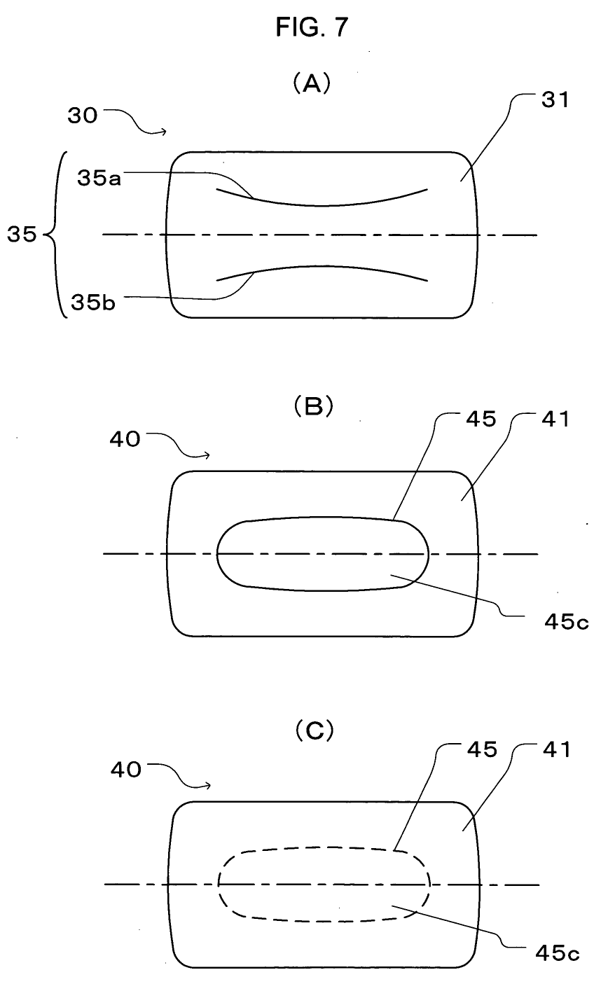

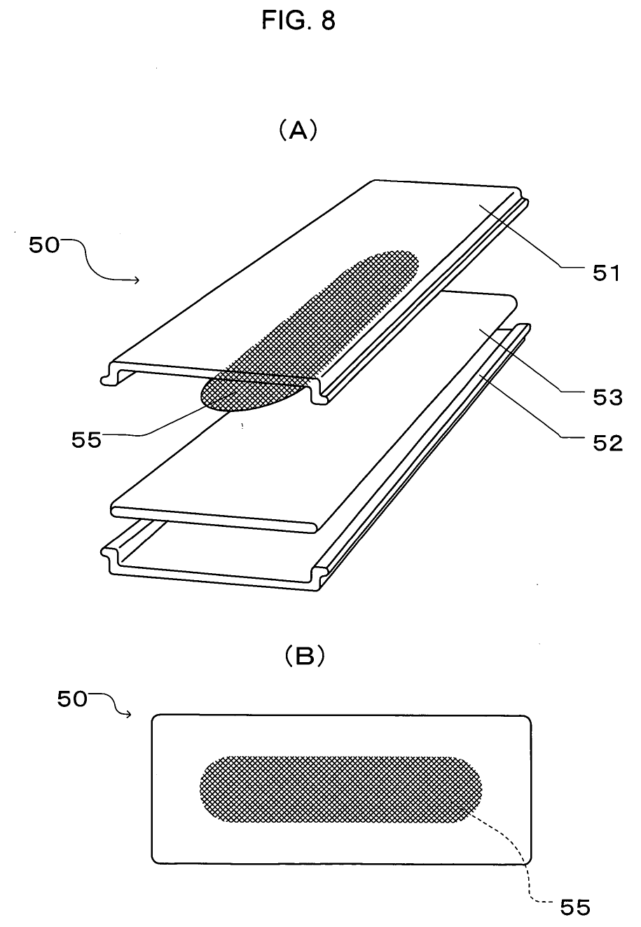

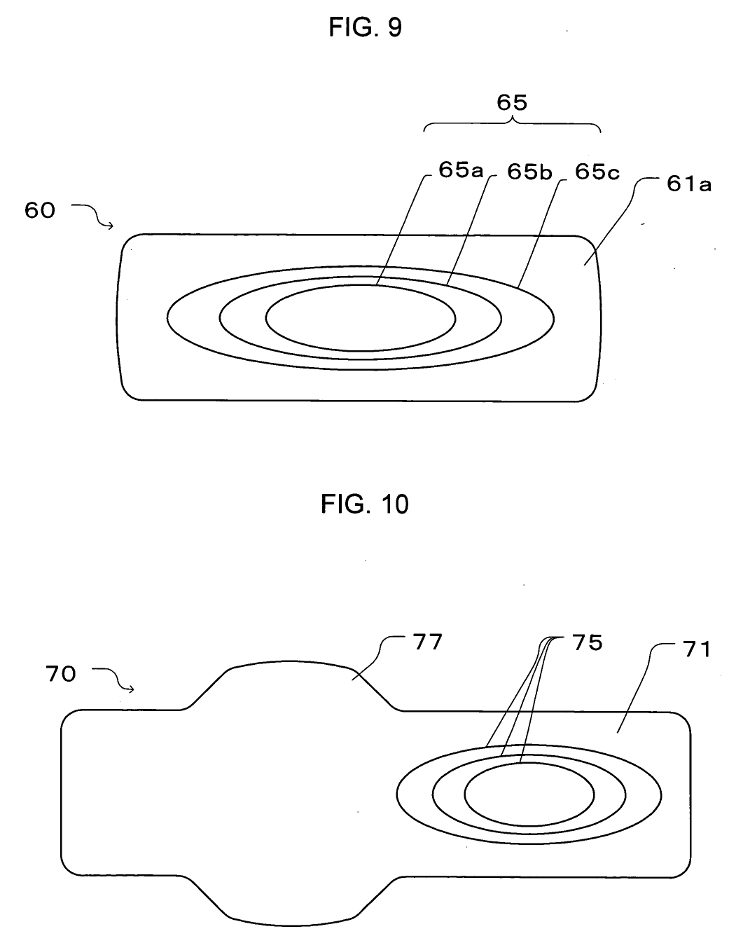

Next, the guide element to be formed on the body side face of the sanitary napkin shall be described. FIG. 7(A) is a top view showing a sanitary napkin 30 where a pair of right and left symmetrical guide elements 35 are formed, FIG. 7(B) is a top view showing a sanitary napkin 40 where a guide element 45 which is a continuous line is formed, and FIG. 7(C) is a top view showing a sanitary napkin 40 where a guide element 45 which is a dashed line is formed. FIG. 8 is an illustrative drawing for illustrating a sanitary napkin 50 provided with a colored sheet having a color different from the surface side sheet 51, FIG. 9 is a top view showing a sanitary napkin 60 where a plurality of guide elements 65 are formed, and FIG. 10 is a top view showing a sanitary napkin 70 with wing 77 where a plurality of guide elements 75 are formed.

<Shape of Guide Element>

In FIG. 7(A), guide elements 45 composed of double lines 35a, 35b that are right and left symmetrical taking the central line in the longitudinal direction as axis are formed on the liquid permeable surface side sheet 31. In this case, an auxiliary pad will be attached by aligning both side edges of the (not shown) auxiliary pad with a pair of double lines 35a, 35b.

On the other hand, in a case where the guide element 45 is formed as a continuous line on the liquid permeable surface side sheet 41, the wearer attach a (not shown) auxiliary pad so as to contain in an area 45c formed inside the guide element 45. Therefore, compared to FIG. 7(A), the attachment position of auxiliary pad is clearer to the wearer.

It should be noted that the guide element 45 is not limited to the continuous line, but it may be a dashed line as in FIG. 7(C) provided that it can shown the area 45c, corresponding the auxiliary pad attachment position.

<Colored Sheet>

In case of composing the guide element by a colored sheet, as shown in FIG. 8(A), a colored sheet 55 colored by a color different from the surface side sheet is arranged between the liquid permeable surface side sheet 51 and an absorbent body 53. In this case, as the absorbent body 51 is white, the colored sheet portion can be recognized visually from the body side face. By doing so, the wearer can apprehend easily the attachment position of an (not shown) auxiliary pad, by the difference in color.

<Plurality of Guide Elements>

A plurality of guide elements can be provided, so that the size or attachment position of the auxiliary pad can be adjusted finely according to the variation of menstrual blood discharge amount or the figure of the wearer.

For instance, for the sanitary napkin 60 shown in FIG. 9, three guide elements 65 are disposed at different positions respectively on the body side face 61a. This guide element 65 is divided into one for small size auxiliary pad 65a, one for medium size auxiliary pad 65b, and one for large size auxiliary pad 65c, allowing to cope with the (not shown) auxiliary pad of three kinds of size.

Besides, in case of having a wing 77 as a sanitary napkin 70 shown in FIG. 10, the wing 77 is formed not in the middle but near the front (portion in contact with the vicinity of the pubic bone of the wearer during the fitting) in the longitudinal direction, and the guide element 75 is provided in a range from the proximity of the anus to the sacrum of the wearer. Thereby, a sanitary product presenting an extremely high prevention effect of backward leakage of menstrual blood is obtained.

[Auxiliary Pad]

<Composition of the Auxiliary Pad>

The auxiliary pad 2 used for the present embodiment is made as enclosed type enveloping, as shown in FIG. 4, an absorbent body 23 with a liquid permeable cover sheet 21, but without limiting to this, it can also be made as enclosed type enveloping the absorbent body and a non-impermeable material with a sheet made of liquid permeable material, pasted type bonding sheets made of liquid permeable material disposed on both faces of the absorbent body each other across the peripheral edge of the absorbent body, pasted type bonding sheets each other across the peripheral edge of the absorbent body, by arranging a sheet made of liquid permeable material disposed on the body side face of the absorbent body and a sheet made of liquid impermeable material disposed on the opposite body side face to body side face of the absorbent body, and so on.

Any auxiliary pad will do, provided that the appearance shape is a substantially longitudinal shape, and the outline shape thereof shall be selected from rectangular, long oval, sandglass type, and so on.

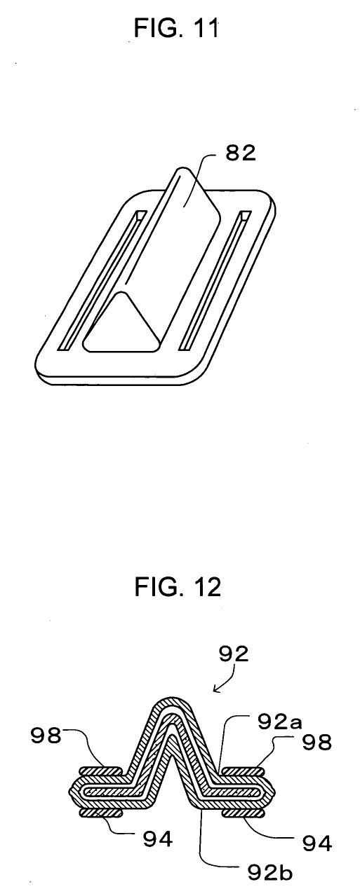

Though the auxiliary pad 2 in the present embodiment is flat shaped, it may also be band like. In case of band-like shape, the cross section shape thereof may be, for instance, rectangular shape, as the auxiliary pad 82 shown in FIG. 11, for facilitating the engagement with the labia.

Next, an auxiliary pad where an adhesive to be pasted to the skin is applied, on the face side in contact with the skin shall be described.

As shown in FIG. 12, an adhesive 94 to attached to a (not shown) sanitary napkin to the vicinity of both side edge portions on a sanitary napkin attachment face 92b of an auxiliary pad 92 and, on the other hand, an adhesive 98 to be pasted to the vicinity of both side edge portions on a skin abutment face 92a. By so doing, a menstrual blood leakage generated by displacement during the wearing ends up to be reduces, because the auxiliary pad 92 and the motion of the wearer act integrally.

[Material of Absorbent Article for Attaching to the Auxiliary Pad and Auxiliary Pad]

<Liquid Permeable Material>

The “liquid permeable material” is preferably a soft material not irritating the labial portion or the labial periphery. For instance, through air non-woven fabric, point bond non-woven fabric obtained by the card method, and so on can be used.

As for other non-woven fabrics, spun lace non-woven fabric obtained by water-flow interlacing treatment is also preferable as it is excellent in drape characteristic. In addition, melt blown non-woven fabric obtained by blowing melt resin into an extremely fine fiber by air pressure and formed into a sheet is also preferable as it is composed of fine and soft fiber.

As for usable fiber material, there are a synthetic bicomponent fiber having a sheath-core structure and a sole synthetic fiber, preferably fiber materials capable of transferring liquid discharged from the interior of the labia to the absorbent body side, profile cross section fibers having Y-shape, C-shape, geometric shape or other cross section shape. Especially, cellulose fibers that are hydrophilic and forming a number of fine grooves are preferable, rayon which is a regenerated fiber, acetate which is a semi-synthetic fiber are preferable. Besides, natural cellulose fibers may also be used. The regenerated cellulose is preferably in a range of 1.1 to 3.3 dtex, the fiber length dimension in a range of 38 to 51 mm, and the natural cellulose fiber is preferably in a range of 5 to 25 mm.

More particularly, laminates formed of fibers where 40 to 90% weight ratio of regenerated cellulose and 10 to 60% weight ratio of natural cellulose fiber are blended are preferable, and a spun lace obtained in a range of 20 to 50 g/m², and more preferably in a range of 25 to 35 g/m² by specific weight per unit by water-flow interlacing treatment can be cited. As another use method, a soft surface material excellent in flexibility to the labial portion can be provided, by adjusting a so-called tow, obtained by refining a continuous fascicule, in a range of 50 to 200 g/m², arranging on the surface side of the product and bonding with a leakage proofing material in a flap area corresponding to the absorbent body, in a way to prevent from dropping. As for other material, a perforated plastic sheet where vent pipes are formed can be used.

<Absorbent Body>

Similarly to the liquid permeable material, the “absorbent body” is preferably a soft material for the labial portion or the labial periphery and, for instance, the following materials can be cited.

Hydrophilic fiber material having liquid retention ability includes wood pulp, natural cellulose fiber, and regenerated cellulose fiber. As for other material, hydrophilic treated hydrophobic synthetic fiber or mixture with a hydrophilic fiber can be cited, and further it may also be a mixture of particulate or fiber-like substance which is a co-called polymer absorbed material selected from acrylic acid, graft polymer of acrylic acid and starch, starch, carboxymethyl cellulose, foam SAP and so on. Otherwise, it may be a mixture of sole or composite synthetic fiber.

Further, as for other material for the absorbent body, sheet or crushed substance selected from tissue, hydrophilic treated foamed sheet, hydrophilic treated melt blown non-woven fabric, fabric web comprising mainly hydrophilic fiber, spun lace non-woven fabric obtained by making fiber web into a sheet, thermal bond non-woven fabric, and so on, airlaid pulp non-woven fabric can be cited.

In consideration of conformity of the shape during the wearing, a foamed form comprising mainly hydrophilic treated synthetic rubber, cellulose sponge comprising mainly cellulose can also be used. Moreover, a so-called tow, obtained by refining bundles of continuous fiber may also be used. Besides, it may also be a laminated material and, for instance, it can also be used from a laminated sheet of a perforated plastic sheet where liquid conduction pipes are formed and a spun lace non-woven or a crushed material of laminated sheet.

As for preferable material of absorbent body, to be more concrete, there is a fiber sheet adjusted in a range of 50 to 500 g/m² and more preferably in a range of 150 to 400 g/m² by stacking one or a plurality of spun lace non-woven fabric(s) obtained as sheet by the water-flow interlacing treatment method from a fiber web where 60 to 90 weight % of regenerated cellulose 2.2 dtex and 10 to 40 weight % of natural cellulose fiber are blended. It becomes possible to improve the shape retention of the absorbent body, by arranging a foam material expanded by 5 to 30 times in a thickness dimension of 1.0 to 10 mm, especially a foam sheet comprising mainly synthetic rubber ingredient, on the bottom face side of a spun lace non-woven fabric.

<Liquid Impermeable Sheet>

The “liquid impermeable sheet” is selected from polyolefin type films using, for instance, LDPE, LLDPE, HDPE as raw material, synthetic rubber type films using, for instance SEBS, SIS, SBS, urethane and so on. It is also possible to use an imperforate vapor permeable film selected from a so-called moisture-permeable film, forming fine cells by blending inorganic filler and applying the drawing treatment and a block copolymer resin of polyester/polyether. Moreover, a perforated moisture-permeable having vent holes of the order not allowing a liquid to leak can be used also.

As for the other material, melt blown non-woven fabric or film, film coated with silicone or other water-repellent agent on one face of a water-soluble which is polyvinyl alcohol can also be used.

<About Leakage Prevention Material>

The leakage prevention material may be used for the absorbent article for attaching to the auxiliary pad and auxiliary pad according to the present invention. The leakage prevention material is selected from soft and liquid impermeable materials. More particularly, it is selected from polyolefin type films using, for instance, LDPE, LLDPE, HDPE as raw material, synthetic rubber type films using, for instance SEBS, SIS, SBS, urethane and so on.

As for the other material, it is also possible to use an imperforate vapor permeable film selected from a so-called moisture permeable film, forming fine cells by blending inorganic filler and applying the drawing treatment and a block copolymer resin of polyester/polyether. Moreover, a perforated moisture-permeable having vent holes of the order not allowing a liquid to leak can be used also.

As for still other material, melt blown non-woven fabric or film, film coated with silicone or other water-repellent agent on one face of a water-soluble which is polyvinyl alcohol can also be used.

<Adhesive>

It is preferable to cover the pressure-sensitive adhesive applied as guided element or pressure-sensitive adhesive applied the skin abutment face, in the auxiliary pad, with a sheet made by coating a foil paper used generally as release paper with silicone resin, or a sheet made by coating a plastic film with silicone resin. By doing so, the pressure adhesion portion is prevented from soiling or peeling off during the storage.

### INDUSTRIAL APPLICABILITY

As described hereinabove, according to the present invention, an auxiliary pad complementing functions of body fluid absorption and so on is made detachable to the body side face of the absorbent article for attaching to the auxiliary pad. Consequently, the absorbent pad can be attached selectively, according to the body fluid (especially menstrual blood) discharge amount.

Moreover, a guide element for guiding the attachment position of the auxiliary pad is formed on the body side face of the absorbent article for attaching to the auxiliary pad and thereby, in case of selecting to attach the auxiliary pad, the wearer can attach the auxiliary pad easily following this guide element. Consequently, it becomes possible to attach the auxiliary pad to an appropriate position in the absorbent article for attaching to the auxiliary pad, allowing to realize a hygienic product offering an excellent prevention effect against body fluid leakage.

1. An absorbent article for attaching to an auxiliary pad for assisting function for absorbing body fluid or other function of the absorbent article, comprising:
a guide element for guiding an attachment position of said auxiliary pad on a body side face directed to a body side of a wearer when a wearer wears the absorbent article.

2. The absorbent article for attaching to an auxiliary pad according to claim 1, wherein said guide element has linear symmetry taking a central line in the longitudinal direction as a symmetrical axis on said absorbent article for attaching to the auxiliary pad.

3. The absorbent article for attaching to an auxiliary pad according to claim 1, wherein said guide element is a continuous line or dashed line of an oval shape having a central line in the longitudinal direction of said absorbent article for attaching to the auxiliary pad as a major axis thereof.

4. The absorbent article for attaching to an auxiliary pad according to claim 1, wherein said guide element comprises a compressed line, a compressed groove, a colored sheet or a printed sheet formed on the body side face of said absorbent article for attaching to the auxiliary pad.

5. The absorbent article for attaching to an auxiliary pad according to 1, wherein said guide element comprises one of the same kind selected from a group consisting of compressed line, compressed groove, colored sheet and
printed sheet formed on the body side face of said absorbent article for attaching to the auxiliary pad, or combination of one or more different kind selected from said group.

6. The absorbent article for attaching to an auxiliary pad according to claim 4, wherein said compressed line has a width dimension ranging from 1.0 to 5.0 mm.

7. The absorbent article for attaching to an auxiliary pad according to claim 4, wherein an interval dimension of a cavity in said compressed groove is 0.5 to 1.5 mm while an interval dimension of a convex portion in said compressed groove is 0.5 to 1.5 mm.

8. The absorbent article for attaching to an auxiliary pad according to claim 4, wherein said colored sheet is enclosed visually recognizably in the absorbent article for attaching to the auxiliary pad.

9. An auxiliary pad to be attached to the absorbent article for attaching to the auxiliary pad according to claim 1, comprising:
a guided element corresponding to the guide element included in said absorbent article for attaching to the auxiliary pad.

10. The auxiliary pad according to claim 9, wherein said auxiliary pad has a band shape or flat shape.

11. The auxiliary pad according to claim 10, wherein said flat shape auxiliary pad is set to have a size so as to generate a surplus in the flat shape auxiliary pad when the guide element included in the absorbent article for attaching to an auxiliary pad is matched with the guided element included in said flat shape auxiliary pad in correspondence to the guide element, and said flat shape auxiliary pad is designed to be attached to said absorbent article for attaching to the auxiliary pad by folding during the use.

12. The auxiliary pad according to claim 11, wherein said flat shape auxiliary pad has a folding guidance element indicating a folding position.

13. The auxiliary pad according to claim 9, wherein an adhesive is applied to a skin abutment face of said auxiliary pad.

14. The auxiliary pad according to claim 9, wherein said auxiliary pad is an auxiliary pad for incontinence of urine.

15. The auxiliary pad according to claim 9, wherein said auxiliary pad is an auxiliary pad for absorbing vaginal discharge.

16. A hygienic product, comprising:
the absorbent article for attaching to an auxiliary pad according to claim 1, and
an auxiliary pad having a guided element corresponding to the guide element included in the absorbent article for attaching to an auxiliary pad, wherein said absorbent article for attaching to the auxiliary pad and said auxiliary pad can be used themselves independently and, at the same time, can be used by freely combining both.

17. The hygienic product of claim 16, wherein said auxiliary pad comprises a plurality of auxiliary pads having different body fluid absorption capacity, and said guide element is provided according to the number of said auxiliary pad.
